(19) United States
(12) Patent Application Publication
(10) Pub. No.: US 2017/0007699 A1
(43) Pub. Date: Jan. 12, 2017

(54) SONOSENSITIZER COMPOSITION CONTAINING TITANIUM OXIDE NANOPARTICLE AS ACTIVE INGREDIENT, COMPOSITION FOR PREVENTING OR TREATING CANCER COMPRISING THE SAME, AND THE PREPARATION THEREOF

(71) Applicant: RESEARCH & BUSINESS FOUNDATION SUNGKYUNKWAN UNIVERSITY, Suwon-si (KR)

(72) Inventors: Jae Hyung PARK, Suwon-si (KR); Dong Gil YOU, Paju-si (KR); Veerasikku Gopal DEEPAGAN, Suwon-si (KR); Min Chang LEE, Gunpo-si (KR)

(73) Assignee: RESEARCH & BUSINESS FOUNDATION SUNGKYUNKWAN UNIVERSITY, Suwon-si (KR)

(21) Appl. No.: 14/797,435

(22) Filed: Jul. 13, 2015

(30) Foreign Application Priority Data
Jul. 10, 2015 (KR) 10-2015-0098709

(51) Int. Cl. A61K 41/00; A61K 9/51; A61N 7/00; A61K 33/24

(52) U.S. Cl. CPC A61K 41/0033; A61K 33/24; A61K 9/5161; A61K 9/5192; A61N 7/00; A61N 2007/0004

(57) ABSTRACT

The present invention relates to a sonosensitizer composition containing titanium dioxide nanoparticles as an active ingredient, a composition for preventing or treating cancer containing the same, and a method of preparing the same. The sonosensitizer of the present invention can solve the problem of the conventional photosensitizer, which can be used only when light can reach the target area, and increase the accumulation level of the sonosensitizer of the present invention to a maximum level within a short time, thereby enabling reduction of the time required for treatment and, due to its human-friendly stability, effective use in cancer treatment.

### TECHNICAL FIELD

The present invention relates to a sonosensitizer composition containing titanium dioxide nanoparticles as an active ingredient, a composition for preventing or treating cancer containing the same, and a method of preparation thereof.

### BACKGROUND OF THE INVENTION

In general, well-known cancer therapies may include removal by surgical operation, radiation therapy, chemotherapy using anticancer agents, etc. However, the conventional therapies such as chemotherapy and surgery have some limitations due to adverse drug reactions (ADR), impairment of host immune system, and poor patient compliance, etc.

Accordingly, energy-based, minimally invasive therapeutic procedures for the destruction of tumors in the natural state have been studied recently, and examples of the minimally invasive therapeutic procedures may include photodynamic therapy (PDT) using lasers, and sonodynamic therapy (SDT), which employs sound (ultrasound) instead of light.

PDT is a method for tumor treatment in which a photosensitizer is injected into the body near the diseased area and laser irradiation applied thereto. PDT has various advantages in that it has few adverse reactions due to a selective treatment on the diseased area, it is a simple operation procedure which can be repeated to increase its therapeutic effect, it can be performed in combination with other therapeutic methods such as surgical operation, radiation therapy, chemotherapy, etc.

However, PDT also has problems in that it allows penetration to a depth of up to 10 mm due to the red light used therein, and it can be applied only to tumor treatment occurring in superficial or local areas in the body. Since currently-available photosensitizers are metabolized at low rates in normal cells, patients should avoid being exposed to light after treatment for 30 days to avoid photosensitive dermatitis. Additionally, PDT requires an arbitrary process to insert optic fibers into the body or tumors, and this increases the pain and risk of the patients, and also due to its inappropriate interactions with biomolecules or aggregation phenomena among photosensitizers, there are limitations in selective delivery of the photosensitizers into the cancer tissues.

Meanwhile, SDT is a non-radiative method which has a low tissue attenuation coefficient, and thus studies on SDT have been progressed based on the mechanism of activating a sonosensitive agent via ultrasonication, which is capable of deep penetration into the live organism and apoptosizing abnormal cells such as tumor cells. Additionally, considering the characteristics of ultrasound enabling deep tissue treatment, SDT may be used in other treatments utilizing thermal effect or HIFU treatments, etc.

Previously, Miller Inc. had developed a derivative of perylenequinone pigment, which is conjugated with a tumor-binding peptide for use in local SDT, but still has not been highlighted.

Accordingly, there is a need for the development of a sonosensitizer that is capable of improving the effect of SDT compared to the conventional products on the market, provides ergonomic-friendly stability, and is widely applicable.

### Prior Art Documents

(Patent Document 1) U.S. Pat. No. 6,627,664

### SUMMARY OF THE INVENTION

### Technical Problem

In order to develop a sonosensitizer with improvement over the conventional products used in the sonodynamic therapy (SDT), the present invention provides a sonosensitizer composition containing titanium dioxide nanoparticles as an active ingredient, a sonosensitizer composition containing the same, and a method of treating cancer using the same.

### Technical Solution

In a first aspect, the present invention provides titanium dioxide nanoparticles, in which the surfaces are modified with a compound represented by Formula 1 below and a polysaccharide,

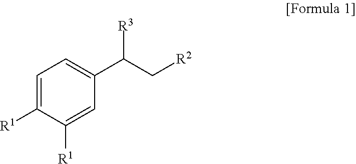

wherein R¹ is —H, —OH, or C₁₋₃ alcohol; R² is —OH, —NH₂, or —NHCH₃; and R³ is —H or —OH.

Preferably, R¹ is —H or —OH; R² is —NH₂ or —NHCH₃; and R³ is —H or —OH.

More preferably, the compound represented by Formula 1 above may be dopamine, norepinephrine, or epinephrine.

The titanium dioxide nanoparticles are reduced by the compound represented by Formula 1 above.

The polysaccharide may be at least one selected from the group consisting of carboxylmethyl dextran, dextran, dextran sulfate, chitosan, hyaluronic acid, hydroxyethyl cellulose, hydroxypropyl cellulose, hydroxyethyl propyl cellulose, guar gum, galactomannan gum, locust bean gum, and starch.

The nanoparticles show a sonosensitizing activity to ultrasound at a frequency ranging from 0.2 MHz to 10 MHz.

The nanoparticles have a negative zeta potential. The nanoparticles may contain titanium in the amount of 1 wt % to 50 wt % based on the total weight of the nanoparticles.

The nanoparticles may have a size ranging from 10 nm to 500 nm.

In a second aspect, the present invention provides a sonosensitizer composition containing titanium dioxide nanoparticles as an active ingredient, in which the surfaces of the titanium dioxide nanoparticles are modified with a compound represented by Formula 1 below and a polysaccharide,

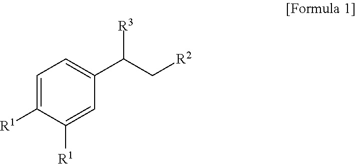

wherein R¹ is —H, —OH, or C₁₋₃ alcohol; R² is —OH, —NH₂, or —NHCH₃; and R³ is —H or —OH.

Preferably, R¹ is —H or —OH; R² is —NH₂ or —NHCH₃; and R¹ is —H or —OH.

More preferably, the compound represented by Formula 1 above may be dopamine, norepinephrine, or epinephrine.

The titanium dioxide nanoparticles are reduced by the compound represented by Formula 1 above.

The polysaccharide may be at least one selected from the group consisting of carboxylmethyl dextran, dextran, dextran sulfate, chitosan, hyaluronic acid, hydroxyethyl cellulose, hydroxypropyl cellulose, hydroxyethyl propyl cellulose, guar gum, galactomannan gum, locust bean gum, and starch.

The sonosensitizer composition shows a sonosensitizing activity to ultrasound at a frequency ranging from 0.2 MHz to 10 MHz.

The sonosensitizer composition has a negative zeta potential.

The sonosensitizer composition may contain titanium in the amount of 1 wt % to 50 wt % based on the total weight of the nanoparticles.

The sonosensitizer composition may have a size ranging from 10 nm to 500 nm.

The sonosensitizer composition may be used in sonodynamic therapy for preventing or treating cancer.

The sonosensitizer composition may be a pharmaceutical composition, and preferably, a pharmaceutical composition for intravenous injection.

The cancer may be at least one selected from the group consisting of breast cancer, lung cancer, stomach cancer, liver cancer, hematomas, bone cancer, pancreatic cancer, brain tumor, skin cancer, thyroid cancer, cutaneous melanoma, ocular melanoma, uterine sarcoma, ovarian cancer, rectal cancer, anal cancer, colorectal cancer, fallopian tube cancer, endometrial cancer, cervical cancer, small bowel cancer, endocrine cancer, thyroid cancer, parathyroid cancer, kidney cancer, soft tissue sarcoma, urethral cancer, prostate cancer, bronchogenic cancer, and bone marrow cancer.

In a third aspect, the present invention provides a sonosensitizer composition including the steps below, or a method of preparing a sonosensitizer composition including:
(1) a first step of dispersing the titanium dioxide nanoparticles into a first solvent, followed by adding the compound represented by Formula 1 below to obtain titanium dioxide nanoparticles, whose surfaces are modified with a compound represented by Formula 1 below; and
(2) a second step of dispersing a polysaccharide in the first solvent, adding a coupling reagent thereto, and then adding the modified titanium dioxide nanoparticles obtained in the first step thereto,

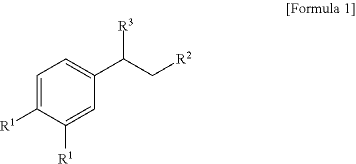

wherein R¹ is —H, —OH, or C₁₋₃ alcohol; R² is —OH, —NH₂, or —NHCH₃; and R³ is —H or —OH.

The first solvent may be at least one selected from the group consisting of formamide, N-methylformamide, dimethyl sulfoxide, and ethylene glycol.

The coupling reagent may be at least one selected from the group consisting of 1-ethyl-3-(3-dimethylaminopropyl)carbodiimide (EDC), N,N′-dicyclohexylcarbodiimide (DCC), N-hydroxysulfosuccinimide (NHS) sulfo-NHS, and 4-dimethylaminopyridine (DMAP).

In a fourth aspect, the present invention provides a method of treating cancer or suppressing cancer metastasis in mammals excluding humans, including administering an effective amount of the sonosensitizer composition.

In a fifth aspect, the present invention provides a method of treating cancer or suppressing cancer metastasis in mammals excluding humans, which includes, after administering an effective amount of the sonosensitizer composition, exposing the mammal to ultrasound to activate the sonosensitizer composition, thereby apoptosizing cancer cells.

### Advantageous Effects

The sonosensitizer of the present invention can not only solve the existing problem in using photosensitizers, which can be used only when light can reach a target area, but also reduce the time for the sonosensitizer to reach the maximum accumulation level, thereby reducing the time required for treatment, and also due to its ergonomic-friendly stability, it can be effectively used for cancer treatment.

### BRIEF DESCRIPTION OF THE DRAWINGS

FIG. 1 shows a schematic diagram illustrating CMD-TiO₂ nanoparticles, which release reactive oxygen species in response to sensing of the ultrasound according to Example 1 of the present invention.

FIG. 2 shows a graph illustrating FT-IR results of CMD-TiO₂ nanoparticles according to Example 1 of the present invention.

FIG. 3 shows a TEM picture of CMD-TiO₂ nanoparticles according to Example I of the present invention.

FIG. 4 shows graphs illustrating the results of an in vitro stability test of CMD-TiO₂ nanoparticles according to Example 1 of the present invention.

FIG. 5 shows graphs illustrating the evaluation results of cytotoxic evaluation of CMD-TiO₂ nanoparticles according to Example 1 of the present invention.

FIG. 6 shows a graph illustrating the evaluation results of release of reactive oxygen species by CMD-TiO₂ nanoparticles according to Example 1 of the present invention.

FIG. 7 shows the pictures and graphs illustrating the evaluation results of tumor accumulation behavior of CMD-TiO₂ nanoparticles according to Example 1 of the present invention.

FIG. 8 shows the pictures and graphs illustrating the evaluation results of in vitro release of reactive oxygen species by CMD-TiO₂ nanoparticles according to Example 1 of the present invention.

FIG. 9 shows the pictures and graphs illustrating the results of therapeutic treatment of tumors using CMD-TiO₂ nanoparticles according to Example 1 of the present invention.

FIG. 10 shows the pictures illustrating the analysis results of tumor tissues after therapeutic treatment using CMD-TiO₂ nanoparticles according to Example 1 of the present invention.

FIG. 11 shows the pictures illustrating the histological analysis results of major organs after therapeutic treatment using CMD-TiO₂ nanoparticles according to Example 1 of the present invention.

FIG. 12 shows the graphs illustrating the detection results of active soluble factors in blood sera and tumor tissues after therapeutic treatment using CMD-TiO₂ nanoparticles according to Example 1 of the present invention.

FIG. 13 shows the pictures illustrating the confirmation results of vascular collapse of tumor tissues after therapeutic treatment using CMD-TiO₂ nanoparticles according to Example 1 of the present invention.

FIG. 14 shows the pictures illustrating the evaluation results of tumor accumulation behavior in a liver tumor-bearing model by CMD-TiO₂ nanoparticles according to Example 1 of the present invention.

FIG. 15 shows the graphs and pictures illustrating the confirmation results of the therapeutic effect of tumor treatment in a liver tumor-bearing model by CMD-TiO₂ nanoparticles according to Example 1 of the present invention.

FIG. 16 shows the pictures illustrating the confirmation results of cancer metastasis after treatment in a liver tumor-bearing model according to Example 1 of the present invention.

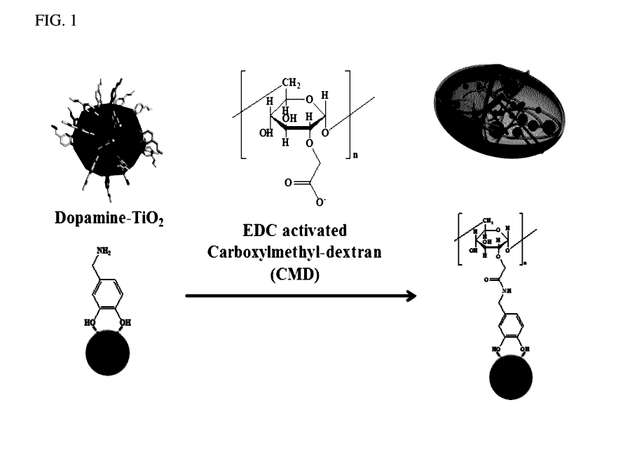

### DETAILED DESCRIPTION

The present invention will be described in more detail with respect to various aspects and exemplary embodiments of the present invention.

In a first aspect, the present invention provides titanium dioxide nanoparticles, in which the surfaces are modified with a compound represented by Formula 1 below and a polysaccharide,

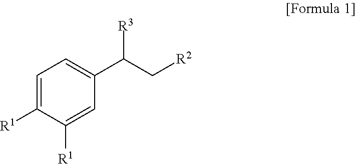

wherein R¹ is —H, —OH, or C₁₋₃ alcohol;

R² is —OH, —NH₂, or —NHCH₃; and

R³ is —H or —OH.

Preferably, R¹ is —H or —OH; R² is —NH₂ or —NHCH₃; and R³ is —H or —OH.

More preferably, the compound represented by Formula 1 above may be dopamine, norepinephrine, or epinephrine.

The titanium dioxide nanoparticles are reduced by the compound represented by Formula 1 above.

Specifically, in the compound represented by Formula 1 above, as the surfaces of the titanium dioxide nanoparticles are reduced, the R¹ group of Formula I is coated on the surfaces of the titanium dioxide nanoparticles, thus making the compound very stable in an aqueous environment. In the case of dopamine, a 1,2-reactive group forms a five-membered chelate with a titanium atom and is coated on the surface of the titanium dioxide nanoparticles. Preferably, the compound represented by Formula 1 may be contained in the amount of 1 wt % to 90 wt % based on the total amount of titanium dioxide.

The polysaccharide may be at least one selected from the group consisting of carboxylmethyl dextran, dextran, dextran sulfate, chitosan, hyaluronic acid, hydroxyethyl cellulose, hydroxypropyl cellulose, hydroxyethyl propyl cellulose, guar gum, galactomannan gum, locust bean gum, and starch.

The polysaccharide is introduced to be coupled with an —NH₂, —OH, or —NHCH₃ reactive group at an end of dopamine, norepinephrine, or epinephrine in order to render titanium dioxide with hydrophilicity. The use of the polysaccharide has advantages in that it can significantly improve long-term circularity and biocompatibility, and can specifically bind to diseased areas.

The nanoparticles show sonosensitizing activity to ultrasound at a frequency from 0.2 MHz to 10 MHz. When the frequency is outside the above range, for example, when the frequency of ultrasound irradiation is lower than 0.2 MHz, there may be a problem in sonosensitization efficiency, whereas when the frequency of ultrasound irradiation is higher than 10 MHz, it may damage normal tissues.

The nanoparticles may be irradiated with ultrasound at a power of 1 W/cm² to 50 W/cm². When the power of ultrasound irradiation is below 1 W/cm², there may be a problem in sonosensitization efficiency, whereas when the power of ultrasound irradiation is higher than 50 W/cm², it may damage normal tissues.

The nanoparticles have a negative zeta potential.

According to the present invention, a zeta potential, which is a surface charge, can be selectively applied to cancer cells only when it is negative. When the zeta potential is positive, it may be applied to all cells, thus causing problems.

Preferably, the zeta potential may be in the range of 0 mV to 50 mV

The nanoparticles may contain titanium in the amount of 1 wt % to 50 wt % based on the total weight of the nanoparticles.

Preferably, the titanium content may be 13 wt % to be within the above range.

When the titanium content is below 1 wt %, there may be a problem in sonosensitization efficiency, whereas when the titanium content exceeds 50 wt %, it may cause a problem in circularity of nanoparticles in an aqueous environment.

According to the present invention, the size of the nanoparticles should be small so that they can arrive at cells after passing through blood vessels, and preferably, the size of the nanoparticles is in the range from 10 nm to 500 nm

Only when the size of the nanoparticles is adjusted to the above range, the nanoparticles can effectively reach cancer cells through the poorly formed walls of blood vessels around a cancer.

In a second aspect, the present invention provides a sonosensitizer composition containing titanium dioxide nanoparticles as an active ingredient, in which the surfaces of the titanium dioxide nanoparticles are modified with a compound represented by Formula 1 below and a polysaccharide.

As used herein, the term “sonosensitizer” exclusively refers to a substance, which, upon exposure to an appropriate frequency, absorbs vibration energy and generates reactive oxygen species, thereby damaging or destroying cells. The sonosensitizer, when introduced into a living organism, can specifically bind to a certain target cell (e.g., a cancer cell), become activated by ultrasound, and produce a toxic substance capable of apoptosizing cancer cells, thereby exhibiting selective toxicity against cancer cells.

From the mechanistic aspect, the present invention is based on the mechanism in which the sonosensitizer can be selectively accumulated in tumor tissues, and bind to oxygen while being activated by ultrasound, thereby generating singlet oxygen (¹O₂), which has extremely high chemical reactivity, and directly damaging cancer cells. In fact, the sonosensitizer of the present invention can not only provide an indirect effect of cancer cell apoptosis by inhibiting nutrient supply to cancer tissues via causing damage on microvessels around a tumor, but can also activate the given immune system by inducing apoptosis or immunological responses by a secondary reaction due to oxidative stress so as to attack cancer tissues, thereby causing necrosis of tumor cells.

In particular, the sonosensitizer of the present invention has solved the drawback in using the conventional light-utilizing photosensitizer, which can be used only when light can reach the target area, and thus can be used for treating all kinds of cancers where ultrasound can reach. Additionally, the sonosensitizer of the present invention can be effectively used in various cancer treatments by increasing its degree of accumulation to a maximum level within a short period of time, thereby reducing time for treatment while providing ergonomic-friendly stability.

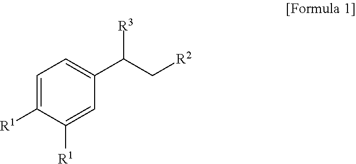

In Formula 1 above, R¹ is —H, —OH, or C₁₋₃ alcohol, R¹ is —OH, —NH₂, or —NHCH₃, and R³ is —H or —OH.

Preferably, R¹ is —H or —OH, R² is —NH₂ or —NHCH₃, R³ is —H or —OH.

More preferably, the compound represented by Formula 1 above may be dopamine, norepinephrine, or epinephrine.

The titanium dioxide nanoparticles are reduced by the compound represented by Formula 1 above.

The polysaccharide may be at least one selected from the group consisting of carboxylmethyl dextran, dextran, dextran sulfate, chitosan, hyaluronic acid, hydroxyethyl cellulose, hydroxypropyl cellulose, hydroxyethyl propyl cellulose, guar gum, galactomannan gum, locust bean gum, and starch.

The sonosensitizer composition shows a sonosensitizing activity to ultrasound at a frequency ranging from 0.2 MHz to 10 MHz.

The sonosensitizer composition has a negative zeta potential.

The sonosensitizer composition may contain titanium in the amount of 1 wt % to 50 wt % based on the total weight of the nanoparticles.

The sonosensitizer composition may have a size ranging from 10 nm to 500 nm.

The sonosensitizer composition may be used in SDT for preventing or treating cancer.

As used herein, the term “sonodynamic therapy (SDT)” refers to a therapeutic method including treating a subject in a morbid state (e.g., a cancer) with a sonosensitizer, and applying photo-irradiation to the subject in order to activate the sonosensitizer to thereby obtain a therapeutic effect.

The sonosensitizer composition may be selectively accumulated in cancer tissues, and the sonosensitizer composition can generate reactive oxygen species, thereby suppressing cancer metastasis.

The sonosensitizer composition may be a pharmaceutical composition, and preferably a pharmaceutical composition for intravenous injection.

The caniers to be included in the pharmaceutical composition of the present invention are those conventionally used in preparing formulations and may include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil, etc., but are not limited thereto. The pharmaceutical composition of the present invention may further include other additives such as lubricants, humectants, sweeteners, flavoring agents, emulsifiers, suspending agents, preservatives, etc., in addition to the components described above.

The pharmaceutical composition of the present invention may be prescribed in various ways considering factors such as formulation method, administration method, age, weight, gender, severity of illness of a patient, diet, administration time, administration route, excretion rate, sensitivity, etc. Preferably, the oral dose of the pharmaceutical composition of the present invention may be 0.0001 mg/kg to 1000 mg/kg (body weight) per day.

The pharmaceutical composition of the present invention may be prepared in a unit dosage form or in a multi-dose container by enclosing the formulation thereinto, using a pharmaceutically acceptable carrier and/or excipient, according to a method by which those skilled in the art to which the present invention belongs can easily practice the invention. In particular, the formulation may be prepared as a solution in oil or an aqueous medium, a suspension or emulsion, or an extract, powder, granules, tablet or capsule, and may further include a dispersing agent or stabilizer.

The cancer may be at least one selected from the group consisting of breast cancer, lung cancer, stomach cancer, liver cancer, hematomas, bone cancer, pancreatic cancer, brain tumor, skin cancer, thyroid cancer, cutaneous melanoma, ocular melanoma, uterine sarcoma, ovarian cancer, rectal cancer, anal cancer, colorectal cancer, fallopian tube cancer, endometrial cancer, cervical cancer, small bowel cancer, endocrine cancer, thyroid cancer, parathyroid cancer, kidney cancer, soft tissue sarcoma, urethral cancer, prostate cancer, bronchogenic cancer, and bone marrow cancer.

In a third aspect, the present invention provides a method for preparing a sonosensitizer composition or a sonosensitizer composition including:
(1) a first step of obtaining titanium dioxide nanoparticles, whose surfaces are modified with a compound represented by Formula 1 below, by dispersing the titanium dioxide nanoparticles into a first solvent, followed by adding the compound represented by Formula 1 below; and
(2) a second step of dispersing a polysaccharide in the first solvent, adding a coupling reagent thereto, and then adding the modified titanium dioxide nanoparticles obtained in the first step thereto,

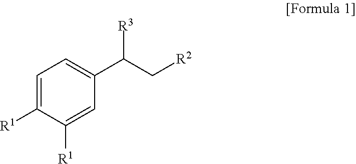

wherein R¹ is —H, —OH, or C₁₋₃ alcohol; R² is —OH, —NH₂, or —NHCH₃; and R³ is —H or —OH.

The first solvent may be at least one selected from the group consisting of formamide, N-methylformamide, dimethyl sulfoxide, and ethylene glycol.

The coupling reagent may be at least one selected from the group consisting of 1-ethyl-3-(3-dimethylaminopropyl)carbodiimide (EDC), N,N-dicyclohexylcarbodiimide (DCC), N-hydroxysulfosuccinimide (NHS) sulfo-NHS, and 4-dimethylaminopyridine (DMAP).

Specifically, the sonosensitizer composition may be prepared by reacting the titanium dioxide nanoparticles, whose surfaces were modified with a compound represented by Formula 1 above by adding the compound represented by Formula 1 after dispersing the titanium dioxide nanoparticles in the first solvent, with a polysaccharide introduced with a leaving group in the presence of the first solvent and the coupling reagent.

The coupling reaction using the coupling reagent may be performed according to a well-known coupling reaction method.

In a fourth aspect, the present invention provides a method of treating cancer or suppressing cancer metastasis in a mammal including administering an effective amount of the sonosensitizer composition to the mammal.

In a fifth aspect, the present invention provides a method of treating cancer or suppressing cancer metastasis in a mammal excluding humans, which includes, after administering an effective amount of the sonosensitizer composition, exposing the mammal to ultrasound to activate the sonosensitizer composition, thereby apoptosizing cancer cells.

The sonosensitizer composition may be administered via intravenous injection.

The sound wave may be an ultrasound having a frequency of 0.2 MHz to 10 MHz, and may be irradiated at a power of 1 W/cm² to 50 W/cm².

The treatment of cancer or suppression of cancer metastasis may be apoptosis of cancer cells by reactive oxygen species generated in response to the sonosensitizer composition.

The mammal refers to mammals excluding humans.

The cancer may be at least one selected from the group consisting of breast cancer, lung cancer, stomach cancer, liver cancer, hematomas, bone cancer, pancreatic cancer, brain tumor, skin cancer, thyroid cancer, cutaneous melanoma, ocular melanoma, uterine sarcoma, ovarian cancer, rectal cancer, anal cancer, colorectal cancer, fallopian tube cancer, endometrial cancer, cervical cancer, small bowel cancer, endocrine cancer, thyroid cancer, parathyroid cancer, kidney cancer, soft tissue sarcoma, urethral cancer, prostate cancer, bronchogenic cancer, and bone marrow cancer.

The principle of SDT is to treat tumors present in the body from outside of the body, as in an ultrasound lithotripsy. After a certain period of time upon injection of the sonosensitizer composition, when ultrasound irradiation is applied to the tumor area, the ultrasound reacts with the sonosensitizer composition accumulated in the tumor cells and thereby selectively removes tumor cells.

Hereinafter, the present invention will be described in detail with reference to the following exemplary embodiments, but these embodiments are disclosed for illustrative purposes only and should not be construed as reducing or limiting the scope of the present invention. Additionally, it should be obvious that those skilled in the art will be able to easily work the invention without specific experimental data, based on the disclosed contents of the present inveniton including exemplary embodiments, and that various modifications and corrections are possible and should also belong to the scope and spirit of the invention.

### EXAMPLE 1

(1) Step 1: Preparation of dopamine-TiO₂ nanoparticles Titanium dioxide (TiO₂, anatase, 10 mg) was dispersed in 5 mL of formamide, slowly charged with 100 μL of dopamine solution (Dopamin, 1.92 μM), and stirred at room temperature. The thus-synthesized dopamine-TiO₂ nanoparticles were washed 3 times with formamide at 13,000 rpm. Finally, the dopamine-Ti₂ nanoparticles were dispersed in 10 mL of formamide.

(2) Step 2: Preparation of dopamine-TiO₂ nanoparticles introduced with carboxylmethyl dextran

The dopamine-TiO₂ nanoparticles were introduced with carboxylmethyl dextran (CMD), which is a biocompatible material.

CMD (200 mg) was dispersed in formamide, charged with 76.7 mg of 1-ethyl-3-(3-dimethylaminopropyl)-carbodiimide (EDC) and 57.5 mg of N-hydroxysulfosuccinimide (NHS), stirred at room temperature for 12 hours, and 2 mL of dopamine-TiO₂ nanoparticles was slowly added thereto. The mixture was stirred at room temperature for 10 hours, charged with 0.1 M NaOH solution, and purified with a 50 kDa membrane using sodium borate buffer (pH 8.6, 4° C.) and distilled water (room temperature) for 48 hours. Upon purification, the resultant was subjected to an ultrasound lithotripter, filtered through a 0.8 μm filter, and freeze-dried.

### EXPERIMENTAL EXAMPLE 1

### Analysis of Characteristics of CMD-TiO₂ Nanoparticles

The CMD-TiO₂ nanoparticles prepared in Example 1 were analyzed via FT-IR,

UV, ICP, and a dynamic scattering device.

TABLE 1

| Substance | CMD-TiO₂ |
|---|---|
| Size (nm) | 198.3 ± 6.34 |
| Zeta potential (mV) | −15.8 |
| Titanium content (%) | 13.0 |

As a result, as shown in Table 1 above, it was confirmed that the nanoparticles have a size of 198 nm, a surface charge of 15.8 mV, and the titanium content contained in the total nanoparticles was shown to be 13 wt %.

### EXPERIMENTAL EXAMPLE 2

### Transmission Electron Microscope (TEM)

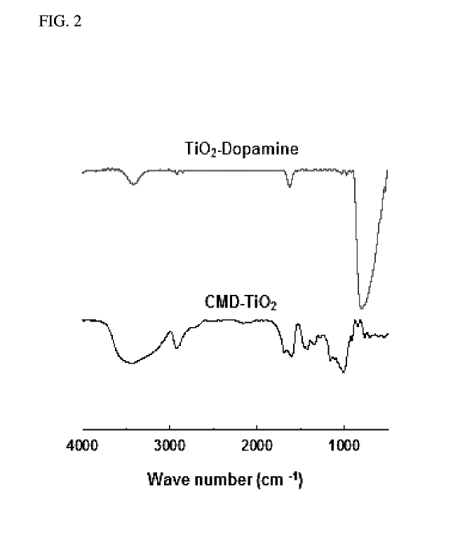

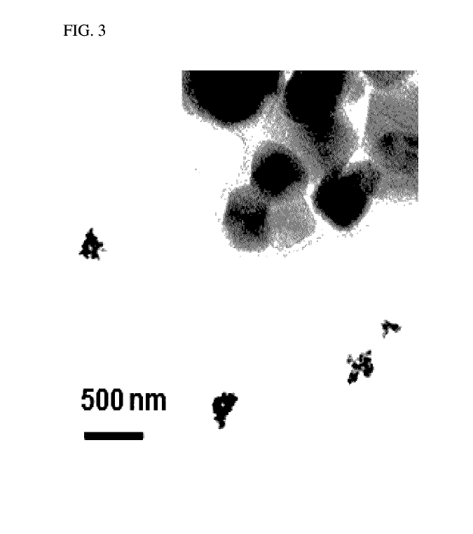

The thus-prepared CMD-TiO₂ nanoparticles were observed under transmission electron microscope (TEM) to examine their morphology, and the surfaces of the titanium particles were shown to be coated with organic materials, as illustrated in FIGS. 2 and 3.

### EXPERIMENTAL EXAMPLE 3

### Validation of In Vitro Stability

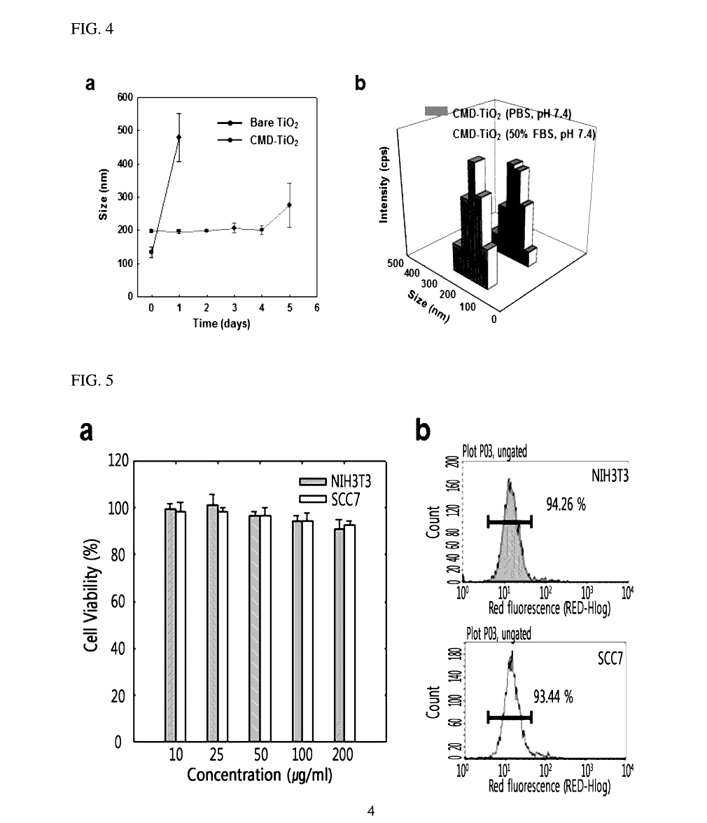

The thus-prepared CMD-TiO₂ nanoparticles were subjected to a stability test to confirm their in vitro stability. As a result, it was confirmed that the CMD-TiO₂ nanoparticles, which were rendered with hydrophilicity, were present in a stable form for at least a few days, as illustrated in FIG. 4. In contrast, the titanium nanoparticles, not introduced with hydrophilicity, were shown to become unstable within a day. Additionally, the CMD-TiO₂ nanoparticles were shown to maintain a size in a stable form in an aqueous solution.

### EXPERIMENTAL EXAMPLE 4

### Evaluation of Cytotoxicity

The thus-prepared CMD-TiO₂ nanoparticles with biocompatibility were subjected to MTT and FACS analyses to examine their cytotoxicity. The results, as illustrated in FIG. 5, confirmed that the CMD-TiO₂ nanoparticles, at concentrations of 10, 25, 50, 100, and 200 μg/mL, did not show any cytotoxicity, in either cancer cells (SCC7) or normal cells (NIH3T3). Additionally, the result of FACS analysis confirmed that the CMD-TiO₂ nanoparticles, at a concentration of 100 μg/mL, did not show any toxicity, in either cancer cells or normal cells.

### EXPERIMENTAL EXAMPLE 5

### Evaluation of Reactive Oxygen Species (ROS) Release

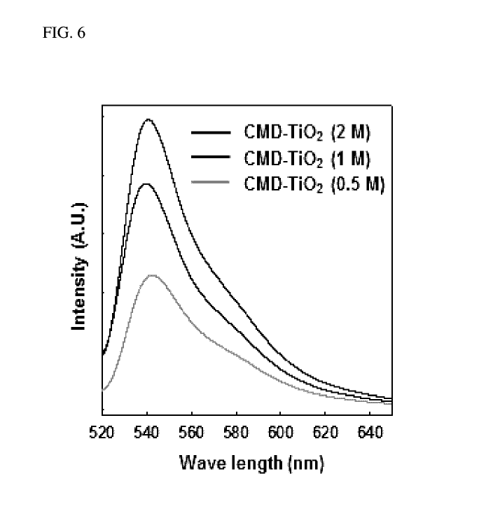

In order to confirm the generation of reactive oxygen species (ROS) from the CMD-TiO₂ nanoparticles in response to high intensity-focused ultrasound via fluorescence quantification, the ROS generated by ultrasound stimulation on the 0.5, 1, and 2 M CMD-TiO₂ nanoparticles was detected using a singlet oxygen detection dye. As a result, it was confirmed that the ROS was released in proportion to the concentration of the given material, as illustrated in FIG. 6.

### EXPERIMENTAL EXAMPLE 6

### Evaluation of Tumor-Accumulation Behavior

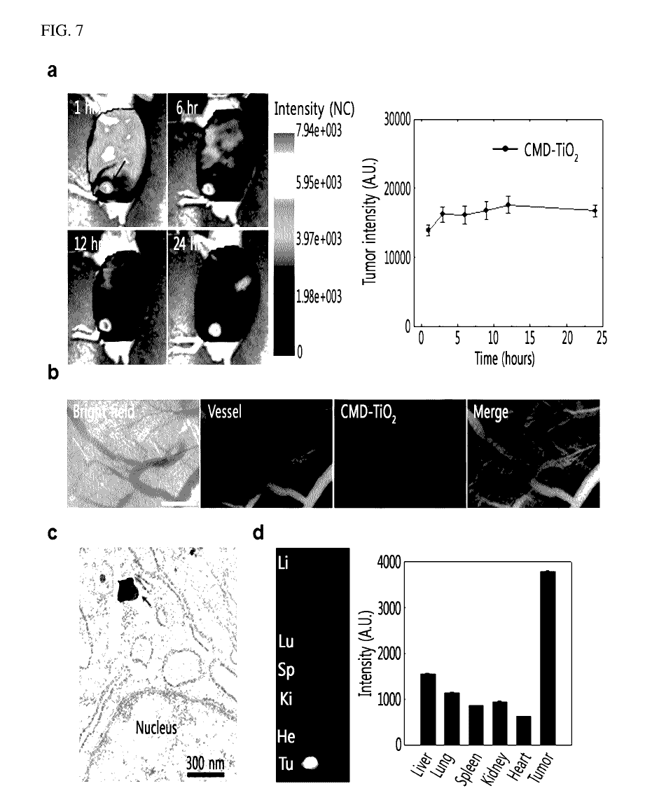

In order to confirm the in vitro behavior of the CMD-TiO₂ nanoparticles with biocompatibility in a tumor-bearing mouse model injected with SCC7 cancer cells, CMD-TiO₂ nanoparticles (5 mg/kg of titanium concentration) were injected intravenously into the mouse, and observed under a fluorescent imaging device for animals. As illustrated in FIG. 7, the result confirmed that the CMD-TiO₂ nanoparticles began to accumulate around the tumor area 1 hour after the injection, were maximally accumulated within 12 hours, and remained accumulated until 24 hours and thereafter. In 24 hours, the tumor-bearing mouse model was sacrificed and the distribution of the CMD-TiO₂ nanoparticles in organs of the animal model was examined. As a result, it was confirmed that the nanoparticles were accumulated at a significantly higher level in the tumor area than in any other organs such as liver, lungs, spleen, kidneys, and heart.

### EXPERIMENTAL EXAMPLE 7

### Evaluation of In Vitro Generation of Reactive Oxygen Species (ROS)

The skin of the tumor area was dissected at the 12 hour time point, when the

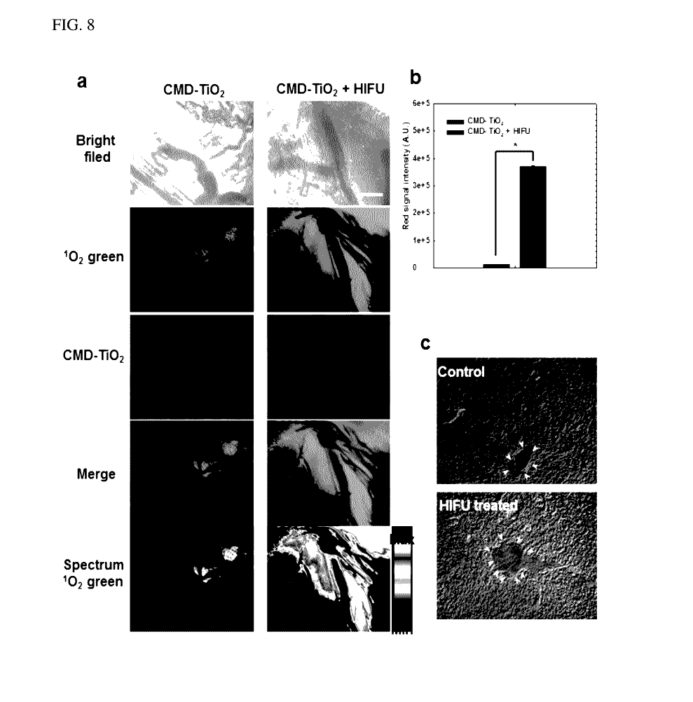

CMD-TiO₂ nanoparticles were maximally accumulated around the tumor area, and the surface of the tumor was observed. As a result, as illustrated in FIG. 8, it was confirmed that the nanoparticles had penetrated into the neighboring tissues from the cancer vessels, and also observation under a Cryo-TEM device revealed that the nanoparticles were present near the cell nuclei.

An in vitro test confirmed that the CMD-TiO₂ nanoparticles generated ROS in response to high intensity-focused ultrasound. In contrast, when a singlet oxygen detection dye was injected into the tumor area after intravenous injection of the CMD-TiO₂ nanoparticles, ROS was shown to be generated only in the tumor tissues treated with ultrasound. Additionally, a quantitative analysis revealed that the ROS generates in the ultrasound-treated tumor was at least 25 times higher than that of the control group.

### EXPERIMENTAL EXAMPLE 8

### Effect of Tumor Treatment

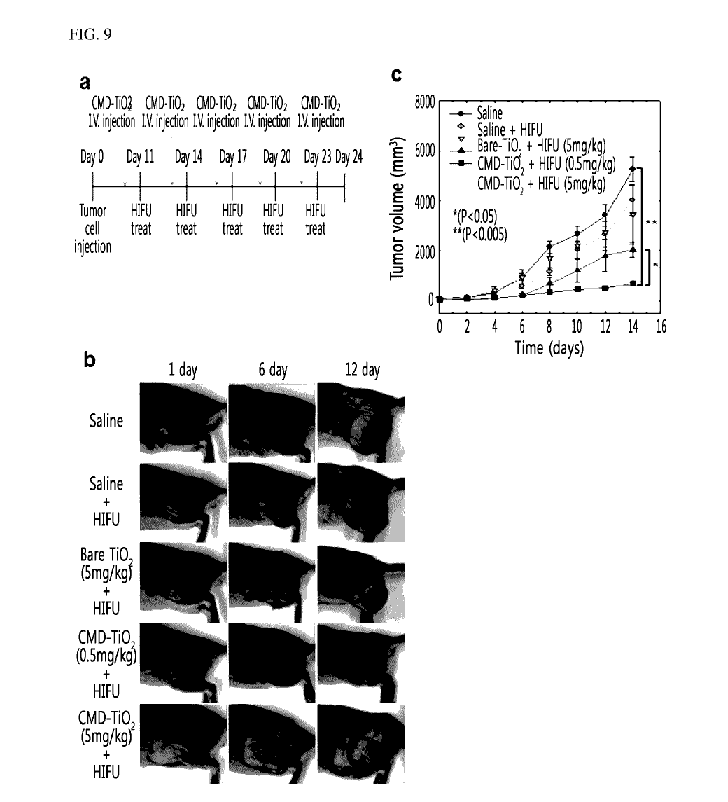

In order to confirm the therapeutic effect of the ROS being released from the CMD-TiO₂ nanoparticles triggered by ultrasound on a tumor-bearing mouse model, the CMD-TiO₂ nanoparticles were intravenously injected at varied titanium concentrations, and the high intensity-focused ultrasound was applied 5 times. As a result, as illustrated in FIG. 9, the therapeutic effect was shown to appear from 0.5 mg/kg of titanium concentration, and at 5 mg/kg of titanium concentration, there was no noticeable increase in the tumor size.

### EXPERIMENTAL EXAMPLE 9

### Histological Analysis of Tumor Tissues According to Tumor Treatment

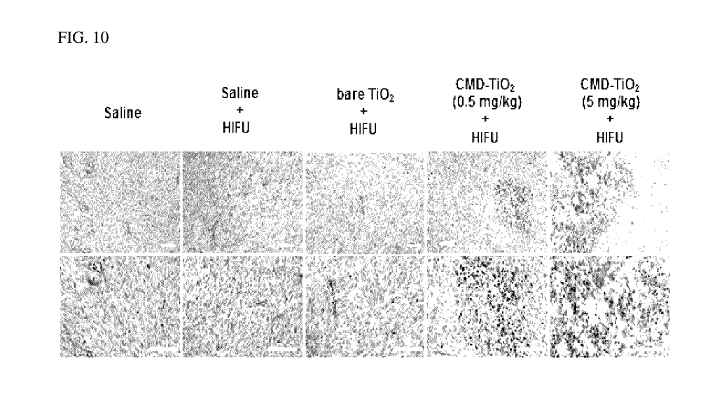

The treated tissues were subjected to histological examination via H&E analysis. As illustrated in FIG. 10, much necrosis was observed in the tumor tissues treated with ultrasound after injecting the CMD-TiO₂ nanoparticles (5 mg/kg of titanium concentration).

### EXPERIMENTAL EXAMPLE 10

### Histological Analysis of Major Organs According to Tumor Treatment

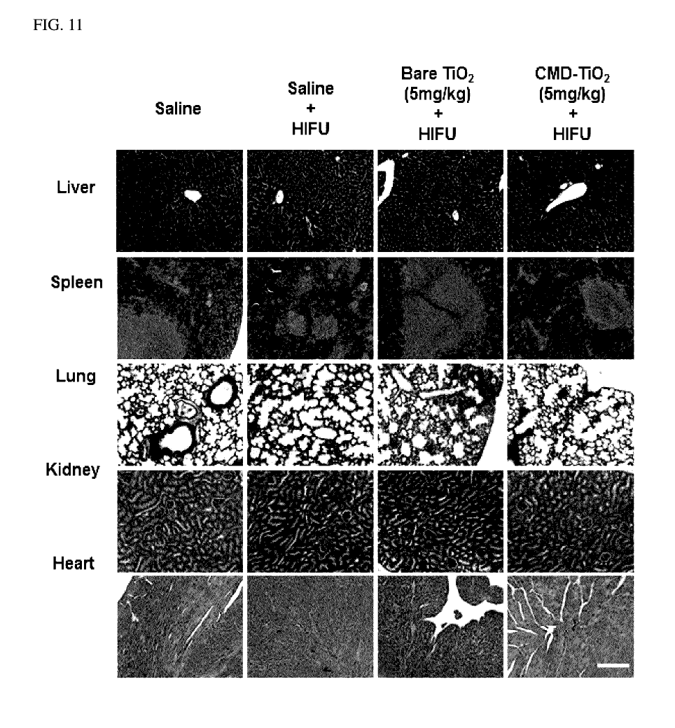

In order to confirm the toxicities in major organs by sacrificing the treated mice, the CMD-TiO₂ nanoparticles (0.5 mg/kg of titanium concentration) were injected intravenously, and the high intensity-focused ultrasound was applied 5 times. As illustrated in FIG. 11, the result confirmed that there was no toxicity observed.

### EXPERIMENTAL EXAMPLE 11

### Confirmation of Detection of Active Soluble Factors in Immune Responses

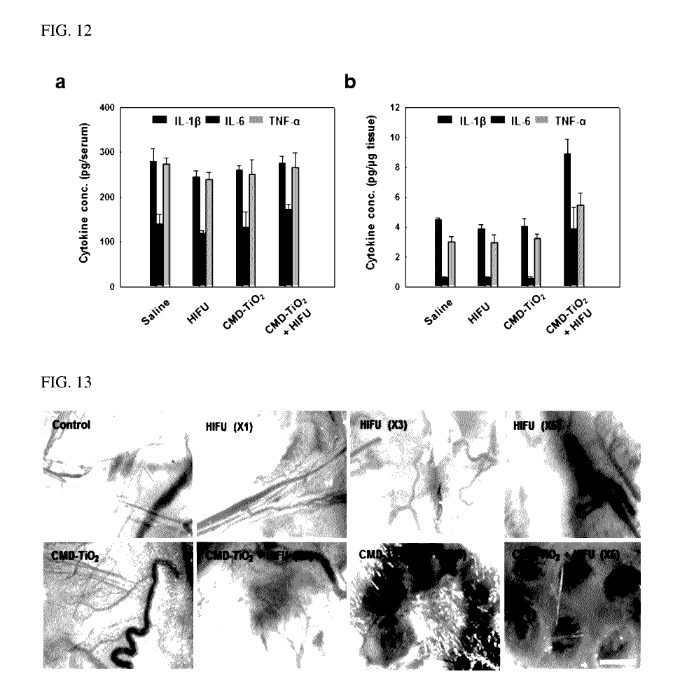

The CMD-TiO₂ nanoparticles (5 mg/kg of titanium concentration) were injected, and the high intensity-focused ultrasound was applied 5 times. Regarding the immune responses of the tumor tissues, after injection with the CMD-TiO₂ nanoparticles followed by ultrasound treatment, as illustrated in FIG. 12, the active soluble factors in blood sera were detected similarly to that of the control group, whereas a significantly higher number of the active soluble factors were detected in the tumor tissues injected with the CMD-TiO₂ nanoparticles followed by ultrasound treatment.

### EXPERIMENTAL EXAMPLE 12

### Confirmation of Vascular Collapse of Tumor Tissues According to Tumor Treatment

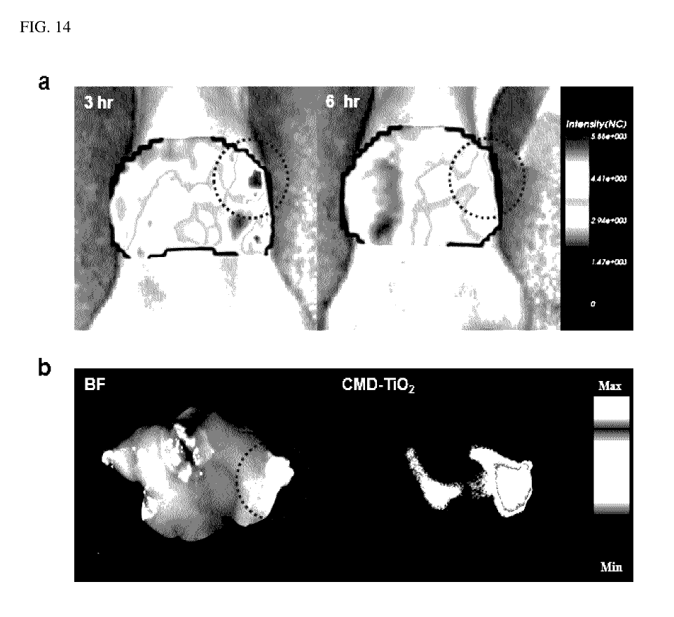

The vascular collapse of tumor tissues, caused by the ultrasound treatment after the injection of the CMD-TiO₂ nanoparticles (5 mg/kg of titanium concentration), was examined. As illustrated in FIG. 13, the result confirmed that the vascular collapse of tumor tissues was shown to appear when the high intensity-focused ultrasound was applied 3 times, and a complete vascular collapse of tumor tissues when the high intensity-focused ultrasound was applied 5 times.

### EXPERIMENTAL EXAMPLE 13

### Evaluation of Liver Tumor Accumulation Behavior

A liver tumor-bearing model was injected with the CMD-TiO₂ nanoparticles and the in vitro accumulation behavior of the nanoparticles was examined. As illustrated in FIG. 14, the result confirmed that the CMD-TiO₂ nanoparticles were effectively accumulated around the liver tumor area, and upon observation of the liver area after sacrificing the mouse, the CMD-TiO₂ nanoparticles were shown to be accumulated only around the tumor area.

### EXPERIMENTAL EXAMPLE 14

### Confirmation of Therapeutic Effect of Liver Tumor Treatment

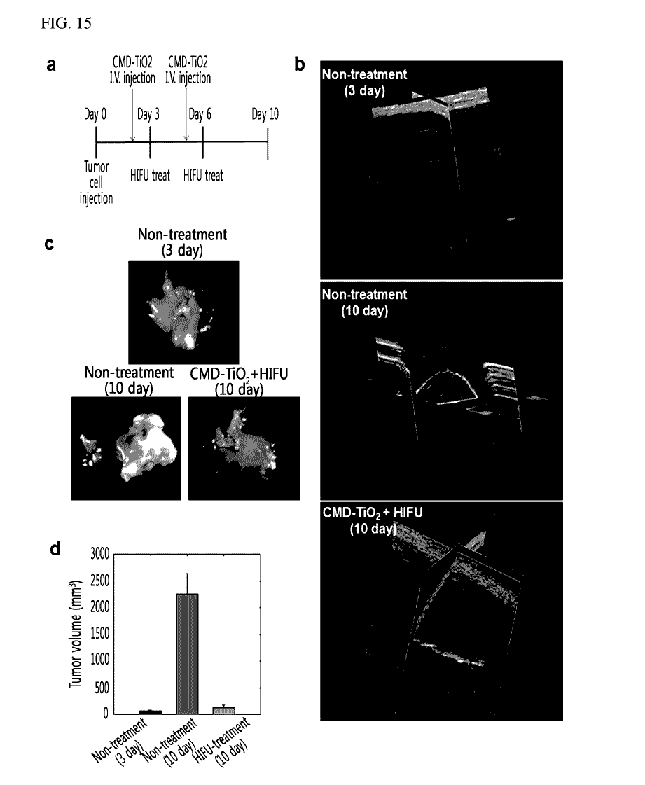

In order to confirm the therapeutic effect of the CMD-TiO₂ nanoparticles on a liver tumor-bearing mouse model, the CMD-TiO₂ nanoparticles were intravenously injected and the high intensity-focused ultrasound was applied twice, and the therapeutic effect was observed. As illustrated in FIG. 15, the result confirmed that the group with the high intensity-focused ultrasound applied twice showed almost no growth in tumor size compared to the control group, which was not treated.

### EXPERIMENTAL EXAMPLE 15

### Evaluation of Cancer Metastasis

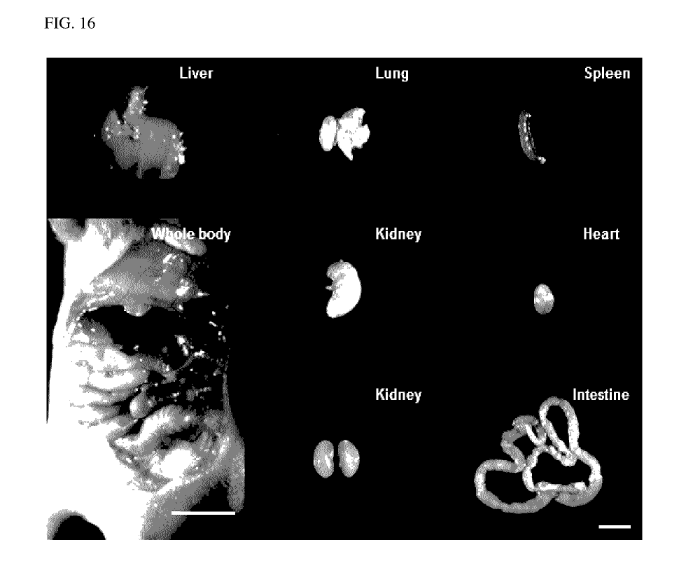

In order to confirm the presence of cancer metastasis by sacrificing the liver tumor-bearing mouse model after the therapeutic treatment, the tumor-bearing mouse was intravenously injected with the CMD-TiO₂ nanoparticles and the high intensity-focused ultrasound treatment was applied twice. As illustrated in FIG. 16, the result confirmed that there was no cancer metastasis in major organs of the mouse.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A titanium dioxide nanoparticle, in which the surface is modified with a compound represented by Formula 1 below and a polysaccharide, 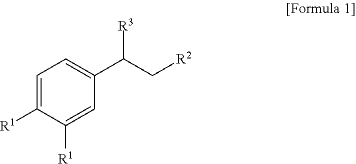
wherein R¹ is —H, —OH, or C₁₋₃ alcohol;
R² is —OH, —NH₂, or —NHCH₃; and
R³ is —H or —OH.

2. The titanium dioxide nanoparticle of claim 1,
wherein R¹ is —H or —OH;
R² is —NH₂ or —NHCH_{3;} and
R³ is —H or —OH.

3. The titanium dioxide nanoparticle of claim 1, wherein the compound represented by Formula 1 above is dopamine, norepinephrine, or epinephrine.

4. The titanium dioxide nanoparticle of claim 1, wherein the titanium dioxide nanoparticle is reduced by a compound represented by Formula 1 below, 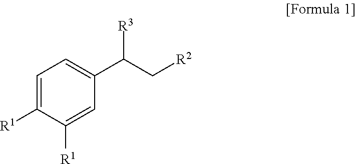
wherein R¹, R², and R³are the same as defined in claim 1.

5. The titanium dioxide nanoparticle of claim 1, wherein the polysaccharide is at least one selected from the group consisting of carboxylmethyl dextran, dextran, dextran sulfate, chitosan, hyaluronic acid, hydroxyethyl cellulose, hydroxypropyl cellulose, hydroxyethyl propyl cellulose, guar gum, galactomannan gum, locust bean gum, and starch.

6. The titanium dioxide nanoparticle of claim 1, wherein the nanoparticle shows a sonosensitizing activity to ultrasound at a frequency ranging from 0.2 MHz to 10 MHz.

7. The titanium dioxide nanoparticle of claim 1, wherein the nanoparticle has a negative zeta potential.

8. The titanium dioxide nanoparticle of claim 1, wherein titanium is contained in the amount of 1 wt % to 50 wt % based on the total weight of the nanoparticle.

9. The titanium dioxide nanoparticle of claim 1, wherein the size of the nanoparticle is from 10 nm to 500 nm.

10. A sonosensitizer composition of claim 1 comprising titanium dioxide nanoparticle as an active ingredient.

11. The sonosensitizer composition of claim 10, wherein the composition is used for preventing or treating cancer.

12. The composition of claim 11, wherein the cancer is at least one selected from the group consisting of breast cancer, stomach cancer, liver cancer, hematomas, bone cancer, pancreatic cancer, brain tumor, skin cancer, thyroid cancer, cutaneous melanoma, ocular melanoma, uterine sarcoma, ovarian cancer, rectal cancer, anal cancer, colorectal cancer, fallopian tube cancer, endometrial cancer, cervical cancer, small bowel cancer, endocrine cancer, thyroid cancer, parathyroid cancer, kidney cancer, soft tissue sarcoma, urethral cancer, prostate cancer, bronchogenic cancer, and bone marrow cancer.

13. The sonosensitizer composition of claim 10, wherein the composition is a pharmaceutical composition.

14. A method of preparing the nanoparticle of claim 1, comprising:
(1) a first step of dispersing the titanium dioxide nanoparticles into a first solvent, followed by adding the compound represented by Formula 1 below, to obtain titanium dioxide nanoparticles, whose surfaces are modified with a compound represented by Formula 1 below; and
(2) a second step of dispersing a polysaccharide in the first solvent, adding a coupling reagent thereto, and then adding the modified titanium dioxide nanoparticles obtained in the first step thereto, 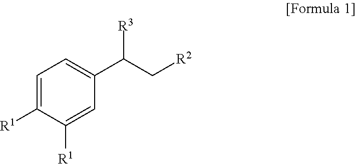
wherein R¹ is —H, —OH, or C₁₋₃ alcohol;
R² is —OH, —NH₂, or —NHCH₃; and
R³ is —H or —OH.

15. The method of claim 14, wherein the first solvent is at least one selected from the group consisting of formamide, N-methylformamide, dimethyl sulfoxide, and ethylene glycol.

16. The method of claim 14, wherein the coupling reagent is at least one selected from the group consisting of 1-ethyl-3-(3-dimethylaminopropyl)carbodiimide (EDC), N,N′-dicyclohexylcarbodiimide (DCC), N-hydroxysulfosuccinimide (NHS), sulfo-NHS, and 4-dimethylaminopyridine (DMAP).

17. A method of treating cancer or suppressing cancer metastasis in a mammal excluding humans, the method comprising administering an effective amount of the sonosensitizer composition of claim 10 to the mammal.

18. A method of treating cancer or suppressing cancer metastasis in a mammal excluding humans, the method comprising, after administering an effective amount of the sono sensitizer composition of claim 10, exposing the mammal to ultrasound to activate the sonosensitizer composition, thereby apoptosizing cancer cells.
